SEQUENCE LISTING (11 sequences)

SEQ ID NO: 1 - DNA, 3093 bp, synthetic construct
  source 1..3093: mol_type = other DNA; note = rice dominant male sterile gene SMS
gtggggactcttcttcgtcctcaccaaacaaaccctagcctcttcataatcggagaaaaagaaagagaaaaccctaaggctctctcccctccggcggcgacaggacgggttctcgatcagggttgccagcgaacacgtctacggtacactcttcgatcgcctcgattctttcttccgattcttcgacgaaatcgcattgtatggccacctaatcttacctgtgtccctttctttctgtgttcttcttcttcatgggcgtcaagatggcggatgaaagcgagcggagcggtatggtaatcgacgacgtcggcggcggactgaatcttccaataattgttgcaggcaagcgtaagcgggagctcacatgggaagagaaggcgttgacagttcttgatattgtcgggtcccaacagcaccccgcatgccaacctgccgagcgtgattgcagcctcatagactcggagaaagattactcatgtgagtgtatatataatccacaactaatcgatcgatcattcatcattcatgtacatctgttctgctttgttcactcgtttgcgtcagtcagtatatccctcttaattggtttttttttcttgtttgttggatgggaaacatcaaaaccaaaactttgcttgttttgcagctatggccggctgccaggccggagaacacgctagtatatttggcattgacaagaatggggatgattctgatgaaccgtgtgctaaggatgatgccaagcaaagtgatgtggctcctctgaaggaggaggagaattgggagctggacagtgaaccagagctcacatgggatgagaaggtggttgaagttctaaacatagttcgacgccgagagatcaccgaatataaccccaagcagttttgctcaattcctactcgattttgtgcctacaacatagccttctttgacctggacaaagagtgtgagtacatccatccatttgtttgttataatatcatccttgttccttgttcattctttgattattgtacatgtcttaaaaaaatttactttcctggtttgatttatgtctctggcttgattttgcagcaaagcttgcacgtggaccacctatcaagtcactagctttccccgactactggtgggagatggactctgtcaatgtgattgcaatcaaggtggccgagtctgatgtgggttaccctatcagggtatttggcactgtgctggccagggatgagtacgatttcaggtgtgtctatttgttcaggcgtgacaggaacaatccacagatcatcacctcgccggtatgttactgattcgttcctcctccttgctagattaatactatttattgttcaaatccatatgccattgccttttcaatccgctgtctgatattctacactataaatgacctgttaattaggctcacattgttaagtgaaatgtggacggttgcatagttccataatggctggtgaaatgcctgatacgatagaaatcctgtttcctggccattaaaatccctactgccagatcttaaaccttgctgtcaatatcatatatccactgaatctgattgcctggtagtccattcctgctgtctaagagggtcaggacagttactgttggaacatcaatcaatccatgacttccccttttttttgtgagatttgatttacttatttagtttctatatcccaagatcctggtacttttacctgttatactattttgttaatccagtgcaacggtagtgctctgggatcatgtacgttttgtactggaaaaaagtggaaacaaaagaatttatattggcatgttgctcatttgaacatgggagtttgaaatgcaattcacaggctgttcataacttcatagacctaaaattattatctgtatgtattatgtacccaaatttctcttaacatattgttggcactgtctgaactgtatttgacttatcgagcctagtgtttctggtggcatttctgacatgattttccctaatctatttataggaggacacactaactctgacaggacctaatcgagcgctgggtgcaatagacaaaatgtatttcgagttcaatttgaagattagagacggtgatgtcgacaaagatttctgcaaaggtgtgcgagaacacaatgccatctgctatacaaagcagccgatgactttatcgctagagagctgcctgagtagaattgattttgtgtatagtcctgttcaattagctgtggaagcttctgttgcagtcaaaattaagggggttgtatccaagtttttcactggcaaagtgactgcttggactactggagatgatcagaacaaaatcatcctgtatgacagtgaagtggaaggcaggaacagagttcttggagctgatggatcagtcgatctgactcgctgtttcgtagccgtcaatttggatgatgaactggtgctcaatgtctgtgtttctgaaggagctggctcaatctttgagctcgttcttggacacaatgatgaagaatgtgtccttgagcaaggtccctatgaactgcaggtgaatgttgtttggacagctgccttgaagcatcgacagcgcagaaaattgtttgagcgtatcggtgacttccgcgtgttaaggtgatatgtagtatgaagatttatatgggggaagctgttacatctcaagcagttctttcttgttatatagattataatttttttgttcagctgagttgaactcgtgcttaactgaaaatctgcctgcaagacttatttagttatatgtactggtagactggtaatctataagattactgatgtctagtagggagtatggatctgtctggtagtctgtataaccttttctgaacctaccaaagtttattttgcgttcttggagaagtgtcttgtctgtcattgttacttactgggttgtgagcatcggattgaggtagtttgtagaggtttgagcgccatgtgtgctctactgttgccatttgctgttgagagatgctgagaacctgacgtttatggctatatatatgttgatgttttgtaagttaatgctactactattgctagaactaagtaagttgattcattttatg

SEQ ID NO: 2 - RNA, 1350 nt, synthetic construct
  source 1..1350: mol_type = other RNA; note = RNA of rice dominant male sterile gene SMS
atggcggatgaaagcgagcggagcggtatggtaatcgacgacgtcggcggcggactgaatcttccaataattgttgcaggcaagcgtaagcgggagctcacatgggaagagaaggcgttgacagttcttgatattgtcgggtcccaacagcaccccgcatgccaacctgccgagcgtgattgcagcctcatagactcggagaaagattactcatctatggccggctgccaggccggagaacacgctagtatatttggcattgacaagaatggggatgattctgatgaaccgtgtgctaaggatgatgccaagcaaagtgatgtggctcctctgaaggaggaggagaattgggagctggacagtgaaccagagctcacatgggatgagaaggtggttgaagttctaaacatagttcgacgccgagagatcaccgaatataaccccaagcagttttgctcaattcctactcgattttgtgcctacaacatagccttctttgacctggacaaagagtcaaagcttgcacgtggaccacctatcaagtcactagctttccccgactactggtgggagatggactctgtcaatgtgattgcaatcaaggtggccgagtctgatgtgggttaccctatcagggtatttggcactgtgctggccagggatgagtacgatttcaggtgtgtctatttgttcaggcgtgacaggaacaatccacagatcatcacctcgccggaggacacactaactctgacaggacctaatcgagcgctgggtgcaatagacaaaatgtatttcgagttcaatttgaagattagagacggtgatgtcgacaaagatttctgcaaaggtgtgcgagaacacaatgccatctgctatacaaagcagccgatgactttatcgctagagagctgcctgagtagaattgattttgtgtatagtcctgttcaattagctgtggaagcttctgttgcagtcaaaattaagggggttgtatccaagtttttcactggcaaagtgactgcttggactactggagatgatcagaacaaaatcatcctgtatgacagtgaagtggaaggcaggaacagagttcttggagctgatggatcagtcgatctgactcgctgtttcgtagccgtcaatttggatgatgaactggtgctcaatgtctgtgtttctgaaggagctggctcaatctttgagctcgttcttggacacaatgatgaagaatgtgtccttgagcaaggtccctatgaactgcaggtgaatgttgtttggacagctgccttgaagcatcgacagcgcagaaaattgtttgagcgtatcggtgacttccgcgtgttaaggtga

SEQ ID NO: 3 - protein, 449 aa, synthetic construct
  source 1..449: note = protein of rice dominant male sterile gene SMS
MADESERSGMVIDDVGGGLNLPIIVAGKRKRELTWEEKALTVLDIVGSQQHPACQPAERDCSLIDSEKDYSSMAGCQAGEHASIFGIDKNGDDSDEPCAKDDAKQSDVAPLKEEENWELDSEPELTWDEKVVEVLNIVRRREITEYNPKQFCSIPTRFCAYNIAFFDLDKESKLARGPPIKSLAFPDYWWEMDSVNVIAIKVAESDVGYPIRVFGTVLARDEYDFRCVYLFRRDRNNPQIITSPEDTLTLTGPNRALGAIDKMYFEFNLKIRDGDVDKDFCKGVREHNAICYTKQPMTLSLESCLSRIDFVYSPVQLAVEASVAVKIKGVVSKFFTGKVTAWTTGDDQNKIILYDSEVEGRNRVLGADGSVDLTRCFVAVNLDDELVLNVCVSEGAGSIFELVLGHNDEECVLEQGPYELQVNVVWTAALKHRQRRKLFERIGDFRVLR

SEQ ID NO: 4 - DNA, 31 bp, synthetic construct
  source 1..31: mol_type = other DNA; note = primer
tctagaatggcggatgaaagcgagcggagcg

SEQ ID NO: 5 - DNA, 31 bp, synthetic construct
  source 1..31: mol_type = other DNA; note = primer
gtcgactcaccttaacacgcggaagtcaccg

SEQ ID NO: 6 - DNA, 19 bp, synthetic construct
  source 1..19: mol_type = other DNA; note = Primer
cgccgatggtttctaccaa

SEQ ID NO: 7 - DNA, 18 bp, synthetic construct
  source 1..18: mol_type = other DNA; note = Primer
ggcgtcggtttccactat

SEQ ID NO: 8 - DNA, 19 bp, synthetic construct
  source 1..19: mol_type = other DNA; note = primer
cctgacggagcgtggttac

SEQ ID NO: 9 - DNA, 20 bp, synthetic construct
  source 1..20: mol_type = other DNA; note = primer
ccagggcgatgtaggaaagc

SEQ ID NO: 10 - DNA, 19 bp, synthetic construct
  source 1..19: mol_type = other DNA; note = primers
ccaacctgccgagcgtgat

SEQ ID NO: 11 - DNA, 19 bp, synthetic construct
  source 1..19: mol_type = other DNA; note = primers
aacttcaaccaccttctca

(19) United States
(12) Patent Application Publication
(10) Pub. No.: US 2024/0336933 A1
(43) Pub. Date: Oct. 10, 2024

(54) RICE DOMINANT MALE STERILE GENE SMS AND APPLICATION THEREOF

(71) Applicants: RICE RESEARCH INSTITUTE, ANHUI ACADEMY OF AGRICULTURAL SCIENCES, HEFEI (CN); UNIVERSITY OF SCIENCE AND TECHNOLOGY OF CHINA, HEFEI (CN); INSTITUTE OF HEALTH AND MEDICINE, HEFEI COMPREHENSIVE NATIONAL SCIENCE CENTER, HEFEI (CN)

(72) Inventors: YACHUN YANG, HEFEI (CN); YONG DING, HEFEI (CN); HAO LI, HEFEI (CN); JIANBO YANG, HEFEI (CN); CHENG ZHANG, HEFEI (CN); HUI MA, HEFEI (CN)

(21) Appl. No.: 18/430,934

(22) Filed: Feb. 2, 2024

(30) Foreign Application Priority Data
Apr. 4, 2023 (CN) 202310352875.7

(51) Int. Cl. C12N 15/82

(52) U.S. Cl. CPC C12N 15/8289; C12N 15/8205

(57) ABSTRACT

A plant sterility-related gene is disclosed, in particular to a dominant male sterility-related gene from rice and application of an encoding protein thereof in creating a rice dominant male sterile line. The present invention provides a rice dominant male sterile gene SMS and application thereof. The rice dominant male sterility-related gene SMS may be used for creating a new dominant male sterile line, thereby strengthening use of the advantages of a rice hybrid. The TO generation of transgenic plant line with overexpression of the rice dominant male sterility related gene SMS shows pollen abortion or no pollen, which indicates that the transgenic plant overexpressing this gene can obtain a new sterile material. Creation of the dominant male sterile line can promote wide application of a recurrent selection breeding method, enrich the genetic diversity of a new breeding variety, and solve the problem of a single variety of the rice.

### REFERENCE TO SEQUENCE LISTING

The substitute sequence listing is submitted as a XML file filed via EFS-Web, with a file name of “Substitute_Sequence_Listing_KIPIUS0114BJZD24.XML”, a creation date of May 2, 2024, and a size of 20,480 bytes. The substitute sequence Listing filed via EFS-Web is a part of the specification and is incorporated in its entirety by reference herein.

### TECHNICAL FIELD

The present invention relates to a plant dominant male sterility-related gene, and in particular to a dominant male sterility-related gene from rice and application thereof in creating a dominant male sterile line.

### BACKGROUND

As a kind of main food crops in China, rice is very important to national food safety. A rice variety (seed) is the source of rice production, and its advantages and disadvantages are related to the safety of the rice industry. From the 1970s to the 1980s, discovery and application of three-line and two-line recessive male sterile materials (Luo et al., 2013) had greatly accelerated the process of hybrid rice breeding, solved the problem of cross-pollination in rice hybridization, and then promoted the second “green revolution” (Zhou et al., 2014). Near 40 years of cross breeding has increased a yield of the rice, but caused the problems of a relatively single genetic resource of the rice, serious homogenization phenomenon and a forceless product due to the fixed genetic background of hybrid parents. Therefore, how to broaden the genetic background of the rice and create new materials with different backgrounds is an urgent task. In recurrent selection breeding, excellent populations are obtained by means of polymerization of excellent genes and traits.

Recurrent selection is an improved hybrid selection method in crop population improvement. Dominant genomic male sterility promotes application of this method. A large number of favorable genes are introduced into dominant genomic male sterile materials to construct outcrossing selection populations, and their hybrid varieties are used for constructing recurrent selection populations. Fertile plants (containing the favorable genes) isolated from their offsprings may be directly used for selecting excellent germplasm resources. This method is conducive to outcrossing within the recurrent selection populations, breaking gene linkages, accelerating recombination and polymerization of the favorable genes, and improving the breeding efficiency. For example, application of Taigu dominant genomic male sterile gene in wheat (Deng Jingyang et al., 1980) is the most successful.

Researches on rice male sterile materials have always been widely concerned. Among them, the research on rice recessive male sterility is deeper, while there are few reports on the research on rice dominant male sterility. The reason may be that there are few rice dominant male sterile resources. As an intermediate material, rice dominant genomic male sterility may be used for rice recurrent selection. Rice dominant genomic male sterility is an important form of male sterility. Cloning and functional analysis of its sterile gene can analyze the genetic mechanism of formation of the rice dominant genomic male sterility, further improve the genetic mechanism of rice male sterility, and also provide a genetic resource and a theoretical basis for application of the rice dominant genomic male sterility at the same time. Whether it is to enrich rice fertility resources or improve the breeding efficiency, it is important and urgent to research the rice dominant genomic male sterile gene.

### SUMMARY

A purpose of the present invention is to isolate a DNA sequence containing a complete encoding segment of a functional protein gene SMS from rice. This gene is used to create a dominant male sterile line, so as to achieve application of this gene in rice recurrent selection breeding.

A first purpose of the present invention is to finely map, clone and apply a gene fragment SMS with the created dominant male sterile line. The SMS gene includes one of the following nucleotide sequences:
1) a DNA sequence shown as SEQ ID No: 1 in a sequence table; or
2) an encoding region sequence of a DNA sequence shown as SEQ ID No: 1, the encoding region sequence being shown as SEQ ID No: 2 in the sequence table, the sequence shown as SEQ ID No: 2 being used for encoding an SMS functional protein and a protein encoded by the DNA sequence shown as SEQ ID No: 1, and the protein including a sequence shown as SEQ ID No: 3 in the sequence table; or
3) subfragments included in 1) and 2).

A second purpose of the present invention is to provide a protein encoded by the rice dominant male sterility-related gene SMS, including an amino acid sequence shown as SEQ ID No: 2.

A third purpose of the present invention is to provide application of the dominant male sterility-related gene SMS in cultivation of a rice dominant male sterile line.

A method for cultivation of the rice dominant male sterile line may include the following steps:
constructing an overexpression vector for the rice dominant male sterility-related gene SMS, transforming the overexpression vector into rice, and performing screening to obtain dominant male sterile transgenic rice.

The overexpression vector is a Ti plasmid vector, preferably, Super1300.

Transformation may use an Agrobacterium-mediated transformation method and a gene gun-mediated transformation method, preferably, the Agrobacterium-mediated transformation method.

For an obtained line overexpressing the dominant male sterile gene SMS, its anthers show no pollen or pollen abortion. The sterile line has normally developed gynoecium stigmas, capable of accepting foreign normal pollens for fertilization and fruiting, and its other nutritive tissue sites can all develop and grow normally.

A host that can be transformed by using an expression vector containing the gene of the present invention may be a variety of crops including the rice, and used for cultivation of dominant male sterile lines of the corresponding crops.

The gene of the present invention is dominant male sterile. Therefore, the gene of the present invention may be combined with a promoter for pollen-specific expression, then inserted into a suitable expression vector, and transformed into the crop host, so that the dominant male sterile line with pollen-specific expression (pollen abortion) may be obtained. There is no significant difference in other nutritive tissue sites between the sterile line and the host.

The present invention provides an important way for creating the dominant male sterile rice. The use of the dominant male sterile line can perform outcrossing pollination and promote the use of the advantages of a hybrid; and the dominant male sterile line can promote application of the recurrent selection breeding method, can enrich the genetic diversity of a breeding offspring, can solve the problem of a single variety at present, can improve the wide adaptability of the rice, and is of great significance to safe production of grains.

| SEQ ID No: 1 | |
|---|---|
| GTGGGGACTCTTCTTCGTCCTCACCAAACAAACCCTAGCCTCTTCATAATCGGAGAAAAA | 60 |
| GAAAGAGAAAACCCTAAGGCTCTCTCCCCTCCGGCGGCGACAGGACGGGTTCTCGATCAG | 120 |
| GGTTGCCAGCGAACACGTCTACGGTACACTCTTCGATCGCCTCGATTCTTTCTTCCGATT | 180 |
| CTTCGACGAAATCGCATTGTATGGCCACCTAATCTTACCTGTGTCCCTTTCTTTCTGTGT | 240 |
| TCTTCTTCTTCATGGGCGTCAAGATGGCGGATGAAAGCGAGCGGAGCGGTATGGTAATCG | 300 |
| ACGACGTCGGCGGCGGACTGAATCTTCCAATAATTGTTGCAGGCAAGCGTAAGCGGGAGC | 360 |
| TCACATGGGAAGAGAAGGCGTTGACAGTTCTTGATATTGTCGGGTCCCAACAGCACCCCG | 420 |
| CATGCCAACCTGCCGAGCGTGATTGCAGCCTCATAGACTCGGAGAAAGATTACTCATGTG | 480 |
| AGTGTATATATAATCCACAACTAATCGATCGATCATTCATCATTCATGTACATCTGTTCT | 540 |
| GCTTTGTTCACTCGTTTGCGTCAGTCAGTATATCCCTCTTAATTGGTTTTTTTTTCTTGT | 600 |
| TTGTTGGATGGGAAACATCAAAACCAAAACTTTGCTTGTTTTGCAGCTATGGCCGGCTGC | 660 |
| CAGGCCGGAGAACACGCTAGTATATTTGGCATTGACAAGAATGGGGATGATTCTGATGAA | 720 |
| CCGTGTGCTAAGGATGATGCCAAGCAAAGTGATGTGGCTCCTCTGAAGGAGGAGGAGAAT | 780 |
| TGGGAGCTGGACAGTGAACCAGAGCTCACATGGGATGAGAAGGTGGTTGAAGTTCTAAAC | 840 |
| ATAGTTCGACGCCGAGAGATCACCGAATATAACCCCAAGCAGTTTTGCTCAATTCCTACT | 900 |
| CGATTTTGTGCCTACAACATAGCCTTCTTTGACCTGGACAAAGAGTGTGAGTACATCCAT | 960 |
| CCATTTGTTTGTTATAATATCATCCTTGTTCCTTGTTCATTCTTTGATTATTGTACATGT | 1020 |
| CTTAAAAAAATTTACTTTCCTGGTTTGATTTATGTCTCTGGCTTGATTTTGCAGCAAAGC | 1080 |
| TTGCACGTGGACCACCTATCAAGTCACTAGCTTTCCCCGACTACTGGTGGGAGATGGACT | 1140 |
| CTGTCAATGTGATTGCAATCAAGGTGGCCGAGTCTGATGTGGGTTACCCTATCAGGGTAT | 1200 |
| TTGGCACTGTGCTGGCCAGGGATGAGTACGATTTCAGGTGTGTCTATTTGTTCAGGCGTG | 1260 |
| ACAGGAACAATCCACAGATCATCACCTCGCCGGTATGTTACTGATTCGTTCCTCCTCCTT | 1320 |
| GCTAGATTAATACTATTTATTGTTCAAATCCATATGCCATTGCCTTTTCAATCCGCTGTC | 1380 |
| TGATATTCTACACTATAAATGACCTGTTAATTAGGCTCACATTGTTAAGTGAAATGTGGA | 1440 |
| CGGTTGCATAGTTCCATAATGGCTGGTGAAATGCCTGATACGATAGAAATCCTGTTTCCT | 1500 |
| GGCCATTAAAATCCCTACTGCCAGATCTTAAACCTTGCTGTCAATATCATATATCCACTG | 1560 |
| AATCTGATTGCCTGGTAGTCCATTCCTGCTGTCTAAGAGGGTCAGGACAGTTACTGTTGG | 1620 |
| AACATCAATCAATCCATGACTTCCCCTTTTTTTTGTGAGATTTGATTTACTTATTTAGTT | 1680 |
| TCTATATCCCAAGATCCTGGTACTTTTACCTGTTATACTATTTTGTTAATCCAGTGCAAC | 1740 |
| GGTAGTGCTCTGGGATCATGTACGTTTTGTACTGGAAAAAAGTGGAAACAAAAGAATTTA | 1800 |
| TATTGGCATGTTGCTCATTTGAACATGGGAGTTTGAAATGCAATTCACAGGCTGTTCATA | 1860 |
| ACTTCATAGACCTAAAATTATTATCTGTATGTATTATGTACCCAAATTTCTCTTAACATA | 1920 |
| TTGTTGGCACTGTCTGAACTGTATTTGACTTATCGAGCCTAGTGTTTCTGGTGGCATTTC | 1980 |
| TGACATGATTTTCCCTAATCTATTTATAGGAGGACACACTAACTCTGACAGGACCTAATC | 2040 |
| GAGCGCTGGGTGCAATAGACAAAATGTATTTCGAGTTCAATTTGAAGATTAGAGACGGTG | 2100 |
| ATGTCGACAAAGATTTCTGCAAAGGTGTGCGAGAACACAATGCCATCTGCTATACAAAGC | 2160 |
| AGCCGATGACTTTATCGCTAGAGAGCTGCCTGAGTAGAATTGATTTTGTGTATAGTCCTG | 2220 |
| TTCAATTAGCTGTGGAAGCTTCTGTTGCAGTCAAAATTAAGGGGGTTGTATCCAAGTTTT | 2280 |
| TCACTGGCAAAGTGACTGCTTGGACTACTGGAGATGATCAGAACAAAATCATCCTGTATG | 2340 |
| ACAGTGAAGTGGAAGGCAGGAACAGAGTTCTTGGAGCTGATGGATCAGTCGATCTGACTC | 2400 |
| GCTGTTTCGTAGCCGTCAATTTGGATGATGAACTGGTGCTCAATGTCTGTGTTTCTGAAG | 2460 |
| GAGCTGGCTCAATCTTTGAGCTCGTTCTTGGACACAATGATGAAGAATGTGTCCTTGAGC | 2520 |
| AAGGTCCCTATGAACTGCAGGTGAATGTTGTTTGGACAGCTGCCTTGAAGCATCGACAGC | 2580 |
| GCAGAAAATTGTTTGAGCGTATCGGTGACTTCCGCGTGTTAAGGTGATATGTAGTATGAA | 2640 |
| GATTTATATGGGGGAAGCTGTTACATCTCAAGCAGTTCTTTCTTGTTATATAGATTATAA | 2700 |
| TTTTTTTGTTCAGCTGAGTTGAACTCGTGCTTAACTGAAAATCTGCCTGCAAGACTTATT | 2760 |
| TAGTTATATGTACTGGTAGACTGGTAATCTATAAGATTACTGATGTCTAGTAGGGAGTAT | 2820 |
| GGATCTGTCTGGTAGTCTGTATAACCTTTTCTGAACCTACCAAAGTTTATTTTGCGTTCT | 2880 |
| TGGAGAAGTGTCTTGTCTGTCATTGTTACTTACTGGGTTGTGAGCATCGGATTGAGGTAG | 2940 |
| TTTGTAGAGGTTTGAGCGCCATGTGTGCTCTACTGTTGCCATTTGCTGTTGAGAGATGCT | 3000 |
| GAGAACCTGACGTTTATGGCTATATATATGTTGATGTTTTGTAAGTTAATGCTACTACTA | 3060 |
| TTGCTAGAACTAAGTAAGTTGATTCATTTTATG | 3093 |
| SEQ ID No: 2 | |
| ATGGCGGATGAAAGCGAGCGGAGCGGTATGGTAATCGACGACGTCGGCGGCGGACTGAAT | 60 |
| CTTCCAATAATTGTTGCAGGCAAGCGTAAGCGGGAGCTCACATGGGAAGAGAAGGCGTTG | 120 |
| ACAGTTCTTGATATTGTCGGGTCCCAACAGCACCCCGCATGCCAACCTGCCGAGCGTGAT | 180 |
| TGCAGCCTCATAGACTCGGAGAAAGATTACTCATCTATGGCCGGCTGCCAGGCCGGAGAA | 240 |
| CACGCTAGTATATTTGGCATTGACAAGAATGGGGATGATTCTGATGAACCGTGTGCTAAG | 300 |
| GATGATGCCAAGCAAAGTGATGTGGCTCCTCTGAAGGAGGAGGAGAATTGGGAGCTGGAC | 360 |
| AGTGAACCAGAGCTCACATGGGATGAGAAGGTGGTTGAAGTTCTAAACATAGTTCGACGC | 420 |
| CGAGAGATCACCGAATATAACCCCAAGCAGTTTTGCTCAATTCCTACTCGATTTTGTGCC | 480 |
| TACAACATAGCCTTCTTTGACCTGGACAAAGAGTCAAAGCTTGCACGTGGACCACCTATC | 540 |
| AAGTCACTAGCTTTCCCCGACTACTGGTGGGAGATGGACTCTGTCAATGTGATTGCAATC | 600 |
| AAGGTGGCCGAGTCTGATGTGGGTTACCCTATCAGGGTATTTGGCACTGTGCTGGCCAGG | 660 |
| GATGAGTACGATTTCAGGTGTGTCTATTTGTTCAGGCGTGACAGGAACAATCCACAGATC | 720 |
| ATCACCTCGCCGGAGGACACACTAACTCTGACAGGACCTAATCGAGCGCTGGGTGCAATA | 780 |
| GACAAAATGTATTTCGAGTTCAATTTGAAGATTAGAGACGGTGATGTCGACAAAGATTTC | 840 |
| TGCAAAGGTGTGCGAGAACACAATGCCATCTGCTATACAAAGCAGCCGATGACTTTATCG | 900 |
| CTAGAGAGCTGCCTGAGTAGAATTGATTTTGTGTATAGTCCTGTTCAATTAGCTGTGGAA | 960 |
| GCTTCTGTTGCAGTCAAAATTAAGGGGGTTGTATCCAAGTTTTTCACTGGCAAAGTGACT | 1020 |
| GCTTGGACTACTGGAGATGATCAGAACAAAATCATCCTGTATGACAGTGAAGTGGAAGGC | 1080 |
| AGGAACAGAGTTCTTGGAGCTGATGGATCAGTCGATCTGACTCGCTGTTTCGTAGCCGTC | 1140 |
| AATTTGGATGATGAACTGGTGCTCAATGTCTGTGTTTCTGAAGGAGCTGGCTCAATCTTT | 1200 |
| GAGCTCGTTCTTGGACACAATGATGAAGAATGTGTCCTTGAGCAAGGTCCCTATGAACTG | 1260 |
| CAGGTGAATGTTGTTTGGACAGCTGCCTTGAAGCATCGACAGCGCAGAAAATTGTTTGAG | 1320 |
| CGTATCGGTGACTTCCGCGTGTTAAGGTGA | 1350 |

SEQ ID No: 3
MADESERSGMVIDDVGGGLNLPIIVAGKRKRELTWEEKALTVLDIVGSQQHPACQPAERD
CSLIDSEKDYSSMAGCQAGEHASIFGIDKNGDDSDEPCAKDDAKQSDVAPLKEEENWELD
SEPELTWDEKVVEVLNIVRRREITEYNPKQFCSIPTRFCAYNIAFFDLDKESKLARGPPI
KSLAFPDYWWEMDSVNVIAIKVAESDVGYPIRVFGTVLARDEYDFRCVYLFRRDRNNPQI
ITSPEDTLTLTGPNRALGAIDKMYFEFNLKIRDGDVDKDFCKGVREHNAICYTKQPMTLS
LESCLSRIDFVYSPVQLAVEASVAVKIKGVVSKFFTGKVTAWTTGDDQNKIILYDSEVEG
RNRVLGADGSVDLTRCFVAVNLDDELVLNVCVSEGAGSIFELVLGHNDEECVLEQGPYEL
QVNVVWTAALKHRQRRKLFERIGDFRVLR*

Except the above-mentioned cultivation of the dominant male sterile line by overexpressing the dominant male sterile gene, the method of introducing anthers (pollens) regulated by the gene SMS in the present invention into cells, tissues, plants, etc. to create the dominant male sterile lines is within the claims of the present invention.

The present invention is further described below in conjunction with the drawings and specific implementations.

### DESCRIPTION OF DRAWINGS

The embodiments of the present invention are described in detail below in combination with the drawings, wherein:

FIG. 1 is a schematic diagram of a result of verifying a constructed SMS overexpression vector of the present invention by enzyme digestion, wherein the SMS overexpression vector is subjected to double digestion with XbaI and SalI, a large fragment represents a linearized Super1300 vector, and a small fragment is an SMS gene fragment.

FIG. 2 shows detection on positive SMS overexpressed plants by PCR, wherein DNA of 30 SMS overexpressed plants is extracted separately, and a hygromycin gene, of about 827 bp in length, in each transgenic plant is detected by PCR, and FIG. 2 shows detection results of some positive plants.

FIG. 3 shows expressions, detected by RT-PCR, of an SMS gene in some TO generation of SMS overexpressed plants, wherein RNA is extracted from anthers of some positive SMS overexpressed plants and reversely transcribed into cDNA; with an expression of SMS in wild type Yangdao 6 as 1, an expression of SMS in each SMS overexpressed plant is detected; and results show that the expression of this gene in each SMS overexpressed plant reaches 22-65 times.

FIG. 4 shows a dominant male sterile line created by overexpressing an SMS gene in rice, wherein A: observation of natural growth phenotypes of wild type Yangdao 6 and transgenic lines; B: observation of pollen staining of wild type Yangdao 6 and the transgenic lines; and results show that pollen abortion occurs after overexpression of the SMS gene.

### DETAILED DESCRIPTION

The present invention is described below with reference to specific embodiments. Those skilled in the art should understand that these embodiments are merely illustrative of the present invention and are not intended to limit the scope of the present invention in any manner.

If not otherwise specified, the experimental methods in the following embodiments are all conventional methods. Raw materials of medicinal materials, reagents, etc. used in the following embodiments are all commercially available products, unless otherwise noted.

### Isolation and Cloning of SMS Gene

By means of population construction, gene mapping and cloning, a dominant male sterile gene SMS was found. Anther RNA was extracted from SMS plants and reversely transcribed into cDNA. A primer was designed according to a sequence of an SMS gene in NCBI (http://www.ncbi.nlm.nih.gov/), and recognition sites of restriction enzymes XbaI and SalI and protective bases were introduced at two ends of the primer respectively. A sequence of the primer is as follows, wherein a TCTAGA base is the recognition site and protective base of the restriction enzyme XbaI; and a GTCGAC base is the recognition site and protective base of the restriction enzyme Sa/l.

XbaI
(SEQ ID No: 4)
FP5′-TCTAGAATGGCGGATGAAAGCGAGCGGAGCG-3′
SalI
(SEQ ID NO: 5)
RP5′-GTCGACTCACCTTAACACGCGGAAGTCACCG-3′

The cDNA obtained by reverse transcription was used as a template for PCR amplification by using a KOD-plus high-fidelity DNA polymerase. The PCR reaction conditions are as follows: pre-denaturation at 94° C. for 3 min, 94° C. for 30 s, 58° C. at 30 s, and 72° C. for 1 min for 35 cycles; and extension at 72° C. for 10 min. An amplified PCR product was inserted into a T vector, and transformed into Escherichia coli XL1-blue; and a resultant was cultured in a constant temperature incubator at 37° C. for 16-18 h. Colonies were selected for PCR and verification by enzyme digestion. A confirmed clone was selected and sent to Beijing Liuhe Huada Gene Technology Co., Ltd. for sequencing analysis to obtain full-length cDNA of a required gene. This clone was named T-SMS.

### Construction of Overexpression Vector for SMS Gene

In order to better analyze a function of SMS, the applicant made it overexpressed in rice, and researched the function of the gene from a phenotype of a transgenic plant.

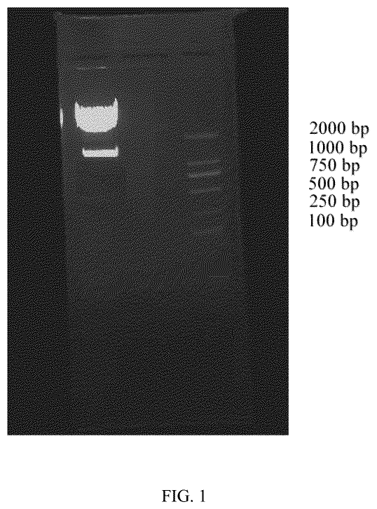

A T-SMS plasmid and an empty vector Super1300 plasmid were subjected to double digestion by restriction enzymes XbaI and SalI, and a target fragment and a large fragment of the Super 1300 plasmid were recovered separately for ligation. A ligase Progema T4 was used to ligate the target fragment to the large fragment of the Super 1300 vector (10×T4 ligase buffer 1 μl, the ligase T4 1 μl, the target fragment 2 μl, and large fragment of the Super 1300 plasmid 6 μl) in a 4° C. refrigerator overnight for 16-18 h, and a ligation product was transformed into Escherichia coli. Clones confirmed by resistance medium screening (a successfully constructed vector only grows on Kana and does not grow on Amp), recombinant screening by colony PCR and verification by enzyme digestion (results are shown in FIG. 1) are sent to Huada Gene Technology Co., Ltd. for sequencing. The clone verified and confirmed by sequencing is a TESLAT1 gene overexpression vector constructed by the present invention, and its plasmid is extracted and transformed into Agrobacterium EHA105. Positive clones were selected, preserved in glycerol at −80° C. and used for rice genetic transformation.

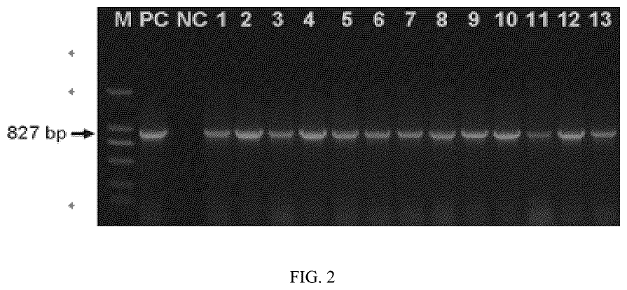

Agrobacterium-Mediated Genetic Transformation of Rice and Identification on Transgenic Plant
(1) Obtaining calli: rice seeds, with no disease spot and well developed embryos, of Yangdao 6 were selected and husked off; the disinfected seeds were soaked in sterile water at 30° C. in the dark overnight; and the embryos were peeled off with a dissecting knife and placed on an induction culture medium. 12 embryos were evenly placed in each dish and placed in dark at 30° C. for 2-3 weeks to induce calli until light yellow granular calli appeared.
(2) Obtaining transgenic plants: the SMS overexpression vector was transformed into the rice calli by using the Agrobacterium-mediated genetic transformation method. The specific transformation process referred to a method published in the literature “An Efficient and High-throughput Protocol for Agrobacterium-mediated Transformation based on Phosphomannose Isomerase Positive Selection in Japonica Rice (Oryza sativa L.). Plant Cell Reports (2012)” by Duan et al. A screening agent used is hygromycin, which is an encoding product of the hygromycin gene contained in the overexpression vector. A total of 30 SMS overexpressed plants were obtained.
(3) The hygromycin gene was amplified by PCR with a Phusion high-fidelity DNA polymerase (produced by NEB Company) by using DNA of the overexpression plants as a template. A total of 13 hygromycin positive plants were detected, as shown in FIG. 2. Primers used by PCR amplification are as follows:

FP:
(SEQ ID No: 6)
5′-CGCCGATGGTTTCTACCAA-3′
RP:
(SEQ ID NO: 7)
5′-GGCGTCGGTTTCCACTAT-3′

### Fertility Screening of T0 Generation of SMS Overexpressed Plants

In order to verify whether the sterility rate of the transgenic plants is increased and whether it is related to the transferred SMS gene, the expressions of the SMS gene in the transgenic rice plants were detected by qRT-PCR. The specific steps are as follows: anthers were collected from the T0 generation of the SMS overexpressed plants in a heading stage (about 95 d after germination) with the wild type Yangdao 6 as a control. The anthers were quickly frozen in liquid nitrogen and stored in an ultra-low temperature refrigerator at −80° C. for RNA extraction, and RNA was reversely transcribed into cDNA. qRT-PCR was performed using a SuperReal fluorescence quantitative premix kit (TIANGEN, SYBR Green, FP205) from Tiangen Company (Beijing). An amount of an RNA template used was quantified by using a rice ACTIN gene as a reference gene. Obtained signals and data were processed by using 2^{-ΔΔCT} (ΔCT=CT target gene−CT reference gene; and ΔΔCT=processed ΔCT−ΔCT control). Three replicates were run for each gene. Quantitative primers for the genes used in this experiment are as follows:

Actin-FP,
(SEQ ID No: 8)
5′-CCTGACGGAGCGTGGTTAC-3′;
Actin-RP,
(SEQ ID NO: 9)
5′-CCAGGGCGATGTAGGAAAGC-3′

For amplification of ACTIN;

SMS-FP,
(SEQ ID NO: 10)
5′-CCAACCTGCCGAGCGTGAT-3′;

SMS-RP, 5′-AACTTCAACCACCTTCTCA-3′ (SEQ ID No:11) used for amplification of the SMS gene.

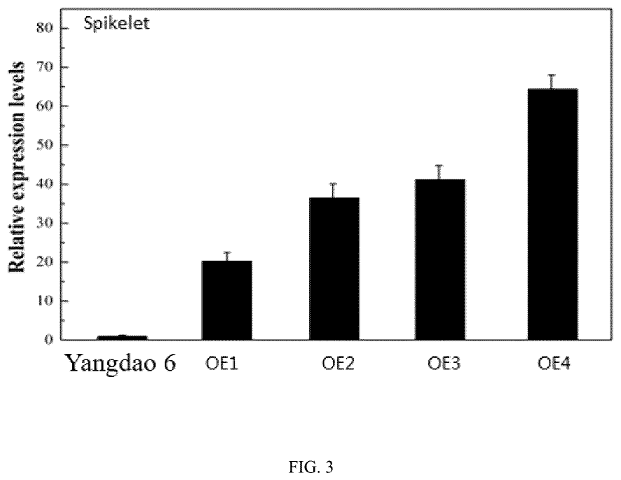

With an expression (CK) of SMS in wild type Yangdao 6 as 1, a result shows that the expression of this gene in each SMS overexpressed plant reaches 22-65 times (FIG. 3).

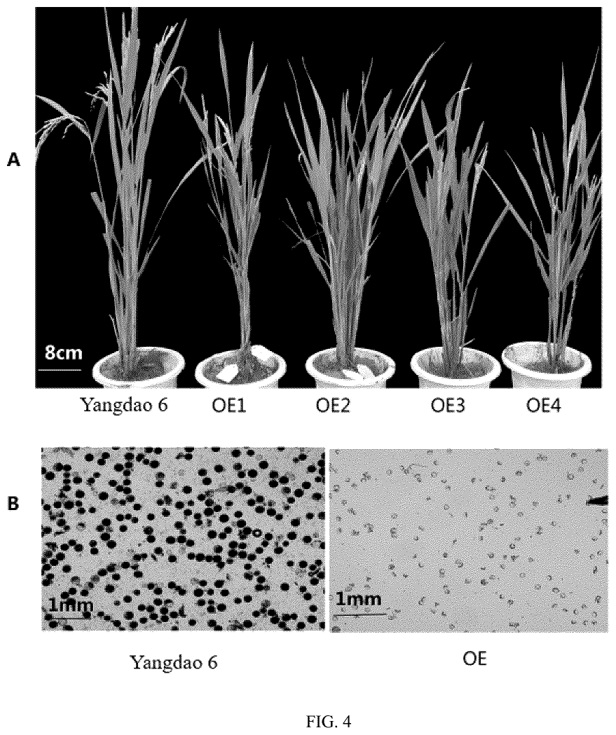

Fertility screening was performed on some families of the T0 generation of plants of the present invention. The specific steps are as follows: a growth status of the rice was observed in a maturity stage of the rice; and results show that the overexpression lines have little difference from the wild type in other phenotypes except for stem wrapping (due to pollen abortion, incomplete spikelet exposure) (FIG. 4A). The pollens of the wild type and the overexpression lines were collected and observed when the rice grew to the heading stage; and results show that the pollens from the wild type develop normally, and the pollens from the overexpression lines are abortive (FIG. 4B). It shows that the SMS gene is indeed related to the plant fertility, and its overexpression can greatly weaken the pollen fertility.

The above description of the specific implementation mode of the present invention is not intended to limit the present invention. Those skilled in the art can make various changes or variations according to the present invention without departing from the spirit of the present invention, and the changes or variations should all belong to the scope of the appended claims of the present invention.

What is claimed is:

1. A rice dominant male sterile gene SMS, extracted from rice and used for creating a rice dominant male sterile line.

2. The rice dominant male sterile gene SMS according to claim 1, comprising a nucleotide sequence shown as at least one of the following:
1) a sequence shown as SEQ ID No: 1 in a sequence table; or
2) an encoding region sequence of a DNA sequence shown as SEQ ID No: 1, the encoding region sequence being shown as SEQ ID No: 2 in the sequence table, the sequence shown as SEQ ID No: 2 being used for encoding an SMS functional protein and a protein encoded by the DNA sequence shown as SEQ ID No: 1, and the protein comprising a sequence shown as SEQ ID No: 3 in the sequence table; or
3) subfragments comprised in 1) and 2).

3. The rice dominant male sterile gene SMS according to claim 1, wherein the sequence of the rice SMS gene is a cDNA sequence or a genomic DNA sequence of the gene, or a DNA sequence having a homology of 90% or higher with the sequences and encoding the same functional protein.

4. An SMS gene capable of creating a dominant male sterile line, encoding the following protein shown as an amino acid sequence (i) or (ii) by using the SMS gene according to claim 1:
(i) a protein encoded by SEQ ID No: 2 in the sequence table;
(ii) a protein derived by substitution, deletion or addition of 1-10 amino acid residues in the amino acid sequence (SEQ ID No: 3) defined in (i), having a function of regulating the plant sterility.

5. A method for creating a dominant male sterile transgenic rice plant, comprising: introducing the rice SMS gene into rice cells, tissues or organs, then cultivating the rice cells, tissues or organs with the rice SMS gene into a plant, and creating a dominant male sterile transgenic rice plant.

6. The method according to claim 5, comprising: introducing the rice SMS gene into plant cells, tissues or organs by means of a plant expression vector.

7. The method according to claim 5, wherein the plant expression vector is Super1300.

8. The method according to claim 5, comprising: adding a constitutive, tissue-specific or inducible promoter in front of a transcribed starting nucleotide when constructing the plant expression vector with the rice SMS gene.

9. The method according to claim 5, comprising: adding the tissue-specific promoter when constructing the plant overexpression vector with the rice SMS gene.
